(19) United States
(12) Patent Application Publication
(10) Pub. No.: US 2019/0343742 A1
(43) Pub. Date: Nov. 14, 2019

(54) CLEANSING COMPOSITION

(71) Applicant: L'OREAL, Paris (FR)

(72) Inventors: Shoibal PATTANAIK, Chembur Mumbai (IN); Amit GHOSH, Chembur Mumbai (IN)

(73) Assignee: L'OREAL, Paris (FR)

(21) Appl. No.: 16/472,033

(22) PCT Filed: Dec. 13, 2017
(86) PCT No.: PCT/EP2017/082718

§ 371 Date: Jun. 20, 2019

(30) Foreign Application Priority Data
Dec. 21, 2016 (IN) 201631043613

(51) Int. Cl. A61K 8/29; A61Q 19/02; A61Q 19/00; A61K 8/36; A61K 8/73; A61K 8/81

(52) U.S. Cl. CPC A61K 8/29; A61Q 19/02; A61K 8/81; A61K 8/36; A61K 8/732; A61Q 19/008

(57) ABSTRACT

The present invention relates to a cleansing composition comprising, in a physiologically acceptable aqueous medium: (a) at least a film-forming starch, (b) at least one cationic polymer, (c) at least one surfactant, (d) at least a soap. The present invention also relates to a process for cleansing the skin, which consists in applying to the said skin said composition, eventually with water, in working the said composition into a foam and then in rinsing off the said composition.

The present invention relates to a cleansing composition which provides skin whitening (skin fairness) and long lasting oil control. This cleansing composition can be particularly used for personal care purposes such as body wash and/or face wash.

Cleansing the skin is very important in the care of the face and/or the body. It must be as efficient as possible since greasy residues, such as excess sebum, the remnants of cosmetic products used daily and make-up products accumulate in the folds of the skin and can block the pores of the skin and result in the appearance of spots. One means for properly cleansing the skin is to use foaming cleansing products.

Skin color is primarily determined by the amount of melanin present in the skin. Thus, cosmetic compositions have been developed to reduce the amount of melanin in the skin and therefore, whiten the skin. These development efforts have focused on whitening agents that inhibit the function and activity of tyrosinase, which plays an important role in the biosynthesis of melanin. However, tyrosinase inhibitors such as hydroquinone, arbutin, kojic acid, among others can induce side effects such as cytotoxicity to melanocytes and potentially mutagenicity to mammalian cells.

Furthermore, oiliness of skin is mainly caused by excess secretion of oily/waxy matter from sebaceous glands which are found in greatest abundance on the face, though they are distributed throughout all skin sites except the palms and soles. Although sebum keeps skin supple, excess sebum can contribute to decrease the skin whitening. Moreover, low sebum level on skin gives a perception of better skin finish or perfect look.

Consequently the challenge is to achieve the completion of two opposite actions through a single rinse-off product, that is to say the washing-off of skin impurities for providing skin whitening and the depositing of whitening agents for long lasting fairness while maintaining the classical properties of a foaming cleanser.

Therefore, there is a need for a cleansing composition which presents both good whitening and oil control properties suitable for the use of face and/or body while providing a good lathering. Advantageously, the needed composition also respects skin. The inventors have demonstrated that the combination of at least a specific starch with at least one cationic polymer, at least a soap and at least one surfactant makes it possible to provide a cleansing composition that advantageously results into skin whitening and long lasting oil control after rinse off.

One object of the present invention is thus a cleansing composition comprising, in a physiologically acceptable aqueous medium:
(a) at least a film-forming starch,
(b) at least one cationic polymer,
(c) at least one surfactant,
(d) at least a soap.

We know from the document CN101564368 that the incorporation of almond and lotus root starch in facial mask products achieves whitening benefits.

Moreover, the document U.S. Pat. No. 6,906,016 describes a personal product liquid cleansing composition comprising a modified or non-modified starch, linear C8 to C13 fatty acids and surfactants which provides good consumer desirable properties while maintaining good stability.

Furthermore, the document U.S. Pat. No. 5,817,609 discloses a skin cleansing bar composition comprising surfactants, a pre-thickened oil and a structuring aid or inert filler selected from fatty acids, water-soluble starches, etc.

However, none of the disclosed compositions achieves both skin whitening and long lasting oil control while providing satisfying quality foaming properties in a single rinse-off product.

As shown in the experimental part, a cleansing composition according to the invention allows to provide a good skin whitening as well as a long lasting oil (sebum) control after rinse off thanks to the presence of a high amylose content in the film-forming starch together with at least one cationic polymer, at least one surfactant and at least a soap.

The cleansing composition according to the invention may be used as a face cleanser as well as hand and/or body cleanser.

According to another aspect, a subject of the present invention is a process for cleansing the skin, which consists in applying to the said skin a composition according to the present invention, eventually with water, in working the said composition into a foam and then in rinsing off the said composition.

The composition according to the invention may be applied directly on wet skin, or alternatively mixed with water and then applied.

During the application of the composition according to the invention on the face and/or the body, the amylose contained in the film-forming starch is deposited on the skin.

After its application on the skin, the cleansing composition according to the invention is rinsed-off (during between 20 seconds and 60 seconds) and then dried. Despite rinsing, a part of the amylose remains deposited on the skin thus forming a uniform white film on the skin which is responsible for long lasting oil control and skin fairness/whiteness.

For the purpose of the present invention, the expression “physiologically acceptable medium” means a medium that is suitable for application of a composition according to the invention on skin.

A physiologically acceptable medium is preferably a cosmetically or dermatologically acceptable medium, that is to say a medium which is devoid of unpleasant odor or appearance and which is entirely compatible with the topical administration route.

Such a medium is in particular considered as physiologically acceptable when it does not cause the user any unacceptable stinging, tautness or redness.

Starch

Starch is a biopolymer that is abundant in nature and inexpensive. It consists in linear α-D-glucan amylose and highly branched amylopectin.

Amylose or amylopectin content in starch depends on its origin. Starches from the conventional sources (e.g., pea, corn, tapioca, potato, etc.) contain less than about 40% by weight of amylose with respect to the total weight of starch.

However, it is possible to increase the amylose content of native starch, for example, by enzymatic debranching (modified) of amylopectin into linear short chain amylose. Such enzyme treated starch may comprise both native long chain amylose and short chain amylose produced by debranching of amylopectin molecules.

As mentioned above, a cleansing composition according to the present invention comprises a film-forming starch.

For the purpose of the present invention, the term “high amylose content in the film-forming starch” is intended to include a starch which contains an amylose content in an amount higher than or equal to 40% by weight, more preferably from 40% to 90%, even more preferably from 45% to 80% by weight, and still more preferably from 50% to 70% by weight with respect to the total weight of starch.

In the present invention, the term “film-forming starch” means a starch suitable, by itself or in the presence of an auxiliary film-forming agent, for forming a macroscopically continuous film that adheres on the skin.

As detailed in the experimental part, by using an aqueous solution comprising 10% by weight of starch (10% solution), film-forming starches (amylose content: 40% and 70%) suitable for the present invention form very transparent and good films (by using BYK Automatic Film Applicator) on polyethylene sheets with high uniformity and homogeneity whereas starches outside of the invention (amylose content: 0%) present a film which is not regular and not uniform.

According to a preferred embodiment, the film-forming property of the starches suitable for the invention is correlated with their viscosity. According to this preferred embodiment, when a starch possesses a viscosity of more than 400 mPa·s (for 10% solution), it is not a film-forming starch suitable for the present invention.

Consequently, in view of the above definitions, among the starches which cannot be considered as film-forming starches suitable for the present invention can be cited, for example, the commercial starches which are sold under the names National 1545® (a modified starch based on waxy maize with added erythorbic acid) and Structure® XL (hydroxypropyl corn starch phosphate, amylose content: 0%) by the company National Starch and Chemical Company and Structure® Zea (hydroxypropyl corn starch phosphate, amylose content: 0%) by the Akzo Nobel company.

As shown in the examples, advantageously, the amylose deposition on skin of a film-forming starch according to the invention increases with the raise of amylose content in the starch.

Thus, the incorporation of film-forming starch in a cleansing composition according to the invention has been found to be effective in terms of whitening delivery after face and/or body wash.

Furthermore, film-forming starches suitable for the present invention compared to the native (unmodified) starch are advantageous for many respects.

Indeed, the film-forming starches suitable for the present invention are easier to implement and possess an interesting rheology.

Typically, the viscosity of the film-forming starches suitable for the present invention, measured at 25° C. and atmospheric pressure, ranges from 20 to 400 mPa·s, preferably from 200 to 400 mPa·s, and more preferably from 200 to 300 mPa·s of an aqueous solution comprising 10% by weight of said starch (10% solution).

The viscosity of the film-forming starch suitable for the present invention may be measured to the here below described protocol for an aqueous solution comprising 10% by weight of said starch.

Viscosity Measurement

In the framework of the present invention, the viscosity measurements are performed at 25° C. and atmospheric pressure using a Rheomat RM180® apparatus.

The sample is brought at the temperature of 25° C.±0.5° C. The viscosity is measured using mobile 2 attachment to Rheomat RM180® at a rotation speed of 200 (fixed) after rotation of the measuring instrument for 10 minutes. The corresponding value in UD (unit deflection) is converted to Pa·s.

Consequently, thanks to their chemical structure and their reduced viscosity, the formulation of film-forming starches suitable for the present invention into cosmetic composition is easier compared to the native starch.

According to a preferred embodiment, a film-forming starch suitable for the present invention is selected from hydroxypropyl pea starch (with an amylose content of 40% by weight with respect to the total weight of starch), hydroxypropyl corn starch modified (with an amylose content of 70% by weight with respect to the total weight of starch), and mixtures thereof.

According to a more preferred embodiment, a film-forming starch suitable for the present invention is selected from hydroxypropyl pea starch (amylose content: 40%) and hydroxypropyl corn starch modified (amylose content: 70%).

For example, among the film-forming starch suitable for the present invention can be used the starches sold under the name of Amaze® Starch by Akzo Nobel Company or under the names of Lycoat® RS 720 and Lycoat® RS 780 by Roquette Company.

According to a still more preferred embodiment, a film-forming starch suitable for the present invention is hydroxypropyl corn starch modified (amylose content: 70%) which may be sold, for example, under the name of Amaze® Starch by Akzo Nobel Company.

In a composition according to the present invention, the film-forming starch is present in an amount from 3% to 12% by weight, preferably in an amount from 4% to 10% by weight, with respect to the total weight of the composition.

Cationic Polymer

The term “cationic polymer” means any polymer comprising cationic groups and/or groups that can be ionized to cationic groups. Preferably, the cationic polymer is hydrophilic or amphiphilic. The preferred cationic polymers are chosen from those that contain units comprising primary, secondary, tertiary and/or quaternary amine groups that may either form part of the main polymer chain or may be borne by a side substituent directly connected thereto

The cationic polymers that may be used preferably have a weight-average molar mass (Mw) of between 500 and 5×106 approximately and preferably between 103 and 3×106 approximately.

Among the cationic polymers, mention may be made more particularly of:

(1) homopolymers or copolymers derived from acrylic or methacrylic esters or amides and comprising at least one of the units of the following formulae:

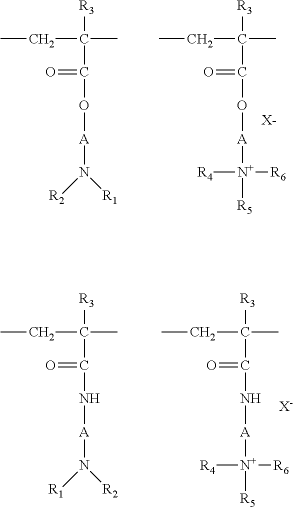

in which:
R₃, which may be identical or different, denote a hydrogen atom or a CH₃ radical;
A, which may be identical or different, represent a linear or branched divalent alkyl group of 1 to 6 carbon atoms, preferably 2 or 3 carbon atoms, or a hydroxyalkyl group of 1 to 4 carbon atoms;
R₄, R₅ and R₆, which may be identical or different, represent an alkyl group containing from 1 to 18 carbon atoms or a benzyl radical, preferably an alkyl group containing from 1 to 6 carbon atoms;
R₁ and R₂, which may be identical or different, represent a hydrogen atom or an alkyl group containing from 1 to 6 carbon atoms, preferably methyl or ethyl;
X denotes an anion derived from a mineral or organic acid, such as a methosulfate anion or a halide such as chloride or bromide.

The copolymers of family (1) may also contain one or more units derived from comonomers that may be selected from the family of acrylamides, methacrylamides, diacetone acrylamides, acrylamides and methacrylamides substituted on the nitrogen with (C₁-C₄)alkyl, acrylic or methacrylic acids or esters thereof, vinyllactams such as vinylpyrrolidone or vinylcaprolactam, and vinyl esters.

Among these copolymers of family (1), mention may be made of:
copolymers of acrylamide and of dimethylaminoethyl methacrylate quaternized with dimethyl sulfate or with a dimethyl halide, such as the product sold under the name Hercofloc by the company Hercules,
copolymers of acrylamide and of methacryloyloxyethyltrimethylammonium chloride, such as those sold under the name Bina Quat® P 100 by the company Ciba Geigy,
copolymers of acrylamide and of methacryloyloxyethyltrimethylammonium methosulfate, such as the product sold under the name Reten® by the company Hercules,
quaternized or non-quaternized vinylpyrrolidone/dialkylaminoalkyl acrylate or methacrylate copolymers, such as the products sold under the name Gafquat® by the company ISP, for instance Gafquat® 734 or Gafquat® 755, or alternatively the products known as Copolymer 845, 958 and 937. These polymers are described in detail in French patents 2 077 143 and 2 393 573,
dimethylaminoethyl methacrylate/vinylcaprolactam/vinylpyrrolidone terpolymers, such as the product sold under the name Gaffix® VC 713 by the company ISP,
vinylpyrrolidone/methacrylamidopropyldimethylamine copolymers, such as those sold under the name Styleze® CC 10 by ISP,
quaternized vinylpyrrolidone/dimethylaminopropylmethacrylamide copolymers such as the product sold under the name Gafquat® HS 100 by the company ISP,
preferably crosslinked polymers of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homopolymerization or copolymerization being followed by crosslinking with an olefinically unsaturated compound, more particularly methylenebisacrylamide. A crosslinked acrylamide/methacryloyloxyethyltrimethylammonium chloride copolymer (20/80 by weight) in the form of a dispersion containing 50% by weight of the said copolymer in mineral oil may be used more particularly. This dispersion is sold under the name Salcare® SC 92 by the company Ciba. A crosslinked methacryloyloxyethyltrimethylammonium chloride homopolymer containing about 50% by weight of the homopolymer in mineral oil or in a liquid ester can also be used. These dispersions are sold under the names Salcare® SC 95 and Salcare® SC 96 by the company Ciba.

(2) Cationic polysaccharides, especially cationic celluloses and galactomannan gums. Among the cationic polysaccharides, mention may be made more particularly of cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups are especially described in French patent 1 492 597, and mention may be made of the polymers sold under the name Ucare Polymer® “JR” (JR 400 LT, JR 125 and JR 30M) or “LR” (LR 400 or LR 30M) by the company Amerchol. These polymers are also defined in the CTFA dictionary as quaternary ammoniums of hydroxyethylcellulose that have reacted with an epoxide substituted with a trimethylammonium group.

Cationic cellulose copolymers or cellulose derivatives grafted with a water-soluble quaternary ammonium monomer are described especially in U.S. Pat. No. 4,131,576, and mention may be made of hydroxyalkylcelluloses, for instance hydroxymethyl-, hydroxyethyl- or hydroxypropylcelluloses grafted, in particular, with a methacryloylethyltrimethylammonium, methacrylamidopropyltrimethylammonium or dimethyldiallylammonium salt. The commercial products corresponding to this definition are more particularly the products sold under the names Celquat® L 200 and Celquat® H 100 by the company National Starch.

The cationic galactomannan gums are described more particularly in U.S. Pat. No. 3,589,578 and 4 031 307, and mention may be made of guar gums comprising cationic trialkylammonium groups. Use is made, for example, of guar gums modified with a 2,3-epoxypropyltrimethylammonium salt (for example, chloride). Such products are especially sold under the names Jaguar® C13 S, Jaguar® C 15, Jaguar® C 17 or Jaguar® C162 by the company Rhodia.

(3) Polymers formed from piperazinyl units and divalent alkylene or hydroxyalkylene radicals containing straight or branched chains, optionally interrupted with oxygen, sulfur or nitrogen atoms or with aromatic or heterocyclic rings, and also the oxidation and/or quaternization products of these polymers.

(4) Water-soluble polyamino amides prepared in particular by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyamino amides can be alkylated or, if they comprise one or more tertiary amine functions, they can be quaternized.

(5) Polyamino amide derivatives resulting from the condensation of polyalkylene polyamines with polycarboxylic acids followed by alkylation with bifunctional agents. Mention may be made, for example, of adipic acid/dialkylaminohydroxyalkyldialkylenetriamine polymers in which the alkyl radical comprises from 1 to 4 carbon atoms and preferably denotes methyl, ethyl or propyl. Among these derivatives, mention may be made more particularly of the adipic acid/dimethylaminohydroxypropyl/diethylenetriamine polymers sold under the name Cartaretine® F, F4 or F8 by the company Sandoz.

(6) Polymers obtained by reacting a polyalkylene polyamine comprising two primary amine groups and at least one secondary amine group with a dicarboxylic acid chosen from diglycolic acid and saturated aliphatic dicarboxylic acids containing from 3 to 8 carbon atoms; the mole ratio between the polyalkylene polyamine and the dicarboxylic acid preferably being between 0.8:1 and 1.4:1; the resulting polyamino amide being reacted with epichlorohydrin in a mole ratio of epichlorohydrin relative to the secondary amine group of the polyamino amide preferably of between 0.5:1 and 1.8:1. Polymers of this type are sold in particular under the name Hercosett® 57 by the company Hercules Inc. or alternatively under the name PD® 170 or Delsette® 101 by the company Hercules in the case of the adipic acid/epoxypropyl/diethylenetriamine copolymer.

(7) Cyclopolymers of alkyldiallylamine or of dialkyldiallylammonium, such as the homopolymers or copolymers containing, as main constituent of the chain, units corresponding to formula (I) or (II):

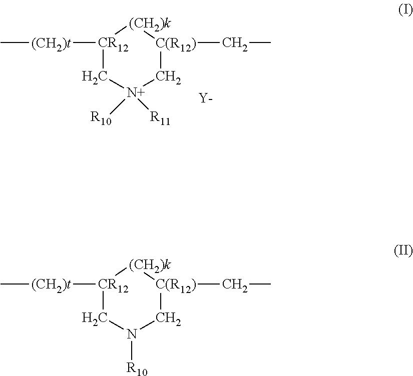

in which:
k and t are equal to 0 or 1, the sum k+t being equal to 1;
R₁₂ denotes a hydrogen atom or a methyl radical;
R₁₀ and R₁₁, independently of each other, denote a C₁-C₆ alkyl group, a hydroxyl(C₁-C₅)alkyl group, a C₁-C₄ amidoalkyl group; or alternatively R₁₀ and R₁₁ may denote, together with the nitrogen atom to which they are attached, an heterocyclic group such as piperidinyl or morpholinyl; R₁₀ and R₁₁, independently of each other, preferably denote a C₁-C₄ alkyl group;
Y— is an anion such as bromide, chloride, acetate, borate, citrate, tartrate, bisulfate, bisulfite, sulfate or phosphate.

Mention may be made more particularly of the dimethyldiallylammonium salt (for example chloride) homopolymer sold for example under the name Merquat® 100 by the company Nalco, and the copolymers of diallyldimethylammonium salts (for example chloride) and of acrylamide, sold especially under the name Merquat® 550 or Merquat® 7SPR.

(8) Quaternary diammonium polymers comprising repeating units of formula:

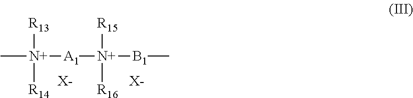

in which:
R₁₃, R₁₄, R₁₅ and R₁₆, which may be identical or different, represent aliphatic, alicyclic or arylaliphatic radicals comprising from 1 to 20 carbon atoms, or C1-C12 hydroxyalkylaliphatic radicals,

or else R₁₃, R₁₄, R₁₅ and R₁₆, together or separately, constitute, with the nitrogen atoms to which they are attached, heterocycles optionally comprising a second non-nitrogen heteroatom,

or else R₁₃, R₁₄, R₁₅ and R₁₆ represent a linear or branched C₁-C₆ alkyl radical substituted with a nitrile, ester, acyl, amide or —CO—O—R₁₇-D or —CO—NH—R₁₇-D group in which R₁₇ is an alkylene and D is a quaternary ammonium group;
A₁ and B₁ represent divalent polymethylene groups comprising from 2 to 20 carbon atoms, linear or branched, saturated or unsaturated, and which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and
X— denotes an anion derived from a mineral or organic acid;

it being understood that A₁, R₁₃ and R₁₅ can form, with the two nitrogen atoms to which they are attached, a piperazine ring;

in addition, if A₁ denotes a linear or branched, saturated or unsaturated alkylene or hydroxyalkylene radical, B₁ may also denote a group (CH₂)n-CO-D-OC—(CH₂)n- in which D denotes:
a) a glycol residue of formula —O—Z—O—, in which Z denotes a linear or branched hydrocarbon-based radical, or a group corresponding to one of the following formulae: —(CH₂—CH₂—O)x-CH₂—CH₂— and —[CH₂—CH(CH₃)—O]y-CH₂—CH(CH₃)—, where x and y denote an integer from 1 to 4, representing a defined and unique degree of polymerization or any number from 1 to 4 representing an average degree of polymerization;
b) a bis-secondary diamine residue such as a piperazine derivative;
c) a bis-primary diamine residue of formula: —NH—Y—NH—, where Y denotes a linear or branched hydrocarbon-based radical, or else the divalent radical —CH₂—CH₂—S—S—CH₂—CH₂—;
d) an ureylene group of formula: —NH—CO—NH—;

Preferably, X— is an anion such as chloride or bromide. These polymers have a number-average molar mass (Mn) generally of between 1000 and 100 000.

Mention may be made more particularly of polymers that are composed of repeating units corresponding to the formula:

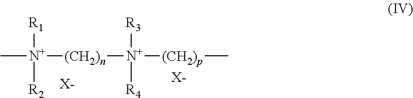

in which R₁, R₂, R₃ and R₄, which may be identical or different, denote an alkyl or hydroxyalkyl radical containing from 1 to 4 carbon atoms, n and p are integers ranging from 2 to 20, and X— is an anion derived from an organic or mineral acid.

A particularly preferred compound of formula (IV) is that for which R₁, R₂, R₃ and R₄ represent a methyl radical and n=3, p=6 and X=Cl, known as Hexadimethrine chloride according to the INCI (CTFA) nomenclature.

(9) Polyquaternary ammonium polymers comprising units of formula (V):

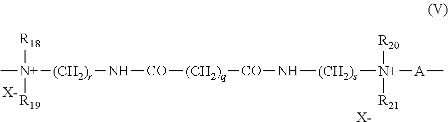

in which:
R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH group, in which p is equal to 0 or to an integer between 1 and 6, with the proviso that R₁₈, R₁₉, R₂₀ and R₂₁ do not simultaneously represent a hydrogen atom,
r and s, which may be identical or different, are integers between 1 and 6,
q is equal to 0 or to an integer between 1 and 34,
X— denotes an anion such as a halide,
A denotes a dihalide radical or preferably represents —CH₂—CH₂—O—CH₂—CH₂—.

Examples that may be mentioned include the products Mirapol® A15, Mirapol® AD1, Mirapol® AZ1 and Mirapol® 175 sold by the company Miranol.

(10) Quaternary polymers of vinylpyrrolidone and of vinylimidazole, for instance the products sold under the names Luviquat® FC 905, FC 550 and FC 370 by the company BASF.

(11) Polyamines such as Polyquart® H sold by Cognis, referred to under the name Polyethylene glycol (15) tallow polyamine in the CTFA dictionary.

(12) Polymers comprising in their structure:

(a) one or more units corresponding to formula (A) below:

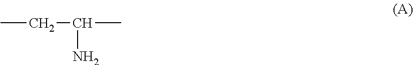

(b) optionally, one or more units corresponding to formula (B) below:

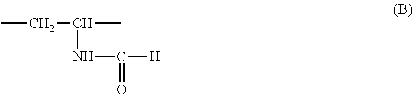

In other words, these polymers may be chosen especially from homopolymers or copolymers comprising one or more units derived from vinylamine and optionally one or more units derived from vinylformamide.

Preferably, these cationic polymers are chosen from polymers comprising, in their structure, from 5 mol % to 100 mol % of units corresponding to formula (A) and from 0 to 95 mol % of units corresponding to formula (B), preferentially from 10 mol % to 100 mol % of units corresponding to formula (A) and from 0 to 90 mol % of units corresponding to formula (B).

These polymers may be obtained, for example, by partial hydrolysis of polyvinylformamide. This hydrolysis may be performed in an acidic or basic medium. The weight-average molecular mass of the said polymer, measured by light scattering, may range from 1000 to 3 000 000 g/mol, preferably from 10 000 to 1 000 000 g/mol and more particularly from 100 000 to 500 000 g/mol.

The polymers comprising units of formula (A) and optionally units of formula (B) are sold especially under the name Lupamin® by the company BASF, for instance, and in a non-limiting manner, the products sold under the names Lupamin® 9095, Lupamin® 5095, Lupamin® 1095, Lupamin® 9030 (or Luviquat® 9030) and Lupamin® 9010.

Other cationic polymers that may be used in the context of the invention are cationic proteins or cationic protein hydrolysates, polyalkyleneimines, in particular polyethyleneimines, polymers comprising vinylpyridine or vinylpyridinium units, condensates of polyamines and of epichlorohydrin, quaternary polyureylenes and chitin derivatives.

Preferably, the cationic polymers are chosen from the polymers of families (1), (2), (7) and (10) mentioned above.

The cationic polymers in the present invention may be chosen from the following polymers:
Polyquaternium 4 (Hydroxyethyl cellulose dimethyl diallylammonium chloride copolymer; Diallyldimethylammonium chloride-hydroxyethyl cellulose copolymer), such as the product Celquat® LOR sold by Akzo Nobel Company;
Polyquaternium 6 (Poly(diallyldimethylammonium chloride)), such as the product Salcare® SC 30 sold by the company BASF, and the product Merquat® 100 sold by the company Nalco (Lubrizol);
Polyquaternium 7 (Copolymer of acrylamide and diallyldimethylammonium chloride), such as the products Merquat® S, Merquat® 2200 and Merquat® 550 sold by the company Nalco (Lubrizol), the product Salcare® SC 10 sold by the company Ciba, the product Mirapol® 550 S BO sold by Rhodia, and the product MERQUAT® 7SPR POLYMER sold by Nalco (Lubrizol);
Polyquaternium 10 (Quaternized hydroxyethyl cellulose) such as the product Polymer JR400® sold by the company Amerchol;
Polyquaternium 11 (Copolymer of vinylpyrrolidone and quaternized dimethylaminoethyl methacrylate) such as the products Gafquat® 755, Gafquat® 755N and Gafquat® 734 sold by the company ISP;
Polyquaternium 16 (Copolymer of methyl vinyl imidazolinium chloride/vinylpyrrolidone) such as the products Luviquat® FC905, Luviquat® FC370, Luviquat® HM552 and Luviquat® FC550 sold by the company BASF;
Polyquaternium 22 (Copolymer of acrylic acid and diallyldimethylammonium Chloride) such as the product Merquat® 280 sold by the company Nalco (Lubrizol);
Polyquaternium 28 (Copolymer of vinylpyrrolidone and dimethylaminopropyl methacrylamide) such as the product Styleze® CC10 sold by the company ISP;
Polyquaternium 32 (Copolymer of Dimethyl Aminoethyl Methacrylate/Acrylamide chloride) such as the product sold by Alfa Chemistry;
Polyquaternium 39 (Terpolymer of acrylic acid, acrylamide and diallyldimethylammonium Chloride), such as the products Merquat® Plus 3330 and Merquat® 3330 PR POLYMER sold by the company Nalco (Lubrizol);
Polyquaternium 46 (Terpolymer of methosulfate of vinylimidazolium/vinylcaprolactame/vinylpyrrolidone), such as the product Luviquat® Hold sold by the company BASF;
Polyquaternium 53 (Terpolymer of acrylic acid/maptac/acrylamide), such as the product Merquat® 2003 P R Polymer sold by the company Lubrizol;
Polyquaternium 67 (Quaternized hydroxyethyl cellulose), such as the product SOFTCAT® POLYMER SL 100 sold by the company Dow Chemical;
Hydroxypropyl guar hydroxypropyl trimonium chloride, such as the product JAGUAR® C 162 sold by the company Rhodia;
and mixtures thereof.

Preferably, the cationic polymers are chosen from Polyquaternium-4, Polyquatemium-7, Polyquatemium-53, Polyquaternium-67, hydroxypropyl guar hydroxypropyl trimonium chloride, and mixtures thereof.

More preferably, the cationic polymers are chosen from Polyquatemium-4, Polyquatemium-7, Polyquaternium-53, Polyquaternium-67, and mixtures thereof.

Still more preferably, the cationic polymers are chosen from Polyquaternium-7, Polyquatemium-67, and mixtures thereof.

The cationic polymer(s) may be in an (active material) amount ranging from 0.3% to 0.6% by weight and better still from 0.4% to 0.5% by weight relative to the total weight of the composition.

The weight ratio of film-forming starch to the cationic polymer may range from 5:1 to 40:1, preferably from 6:1 to 35:1, more preferably from 7:1 to 30:1.

As demonstrated in the examples, the incorporation of a cationic polymer suitable for the present invention has been found to enhance the deposition of amylose starch through a cleansing composition according to the present invention.

Thus, in the present invention, the cationic polymer acts as an amylose deposition enhancer on the skin.

Surfactant

The cleansing composition according to the invention comprises one or more surfactants.

The surfactants may be chosen among anionic surfactants, amphoteric (or zwitterionic) surfactants, nonionic surfactants, and mixtures thereof.

According to a preferred embodiment, the surfactants may be chosen among anionic, nonionic surfactants, and mixtures thereof

Anionic Surfactants

The composition according to the invention may also comprise one or more anionic surfactants.

The term anionic surfactant means a surfactant having only anionic groups as ionic or ionizable groups.

In the present description, an entity is qualified as “anionic” when it has at least one permanent negative charge or when it can be ionized by a negatively charged entity, under the conditions of use of the composition of the invention (medium, pH, for example) and containing no cationic charge.

The anionic surfactants may be sulfate(s) or sulfonate(s) which have at least one sulfate group (—OSO₃H or —OSO₃⁻), and/or a sulfonate group (—SO₃H or —SO₃), or else carboxylic or carboxylate surfactants having at least one carboxylic acid group (—COOH or —COO⁻).

It is understood that the anionic carboxylate surfactants may include one or more sulfate or sulfonate groups; sulfonate anionic surfactants may optionally further comprise one or more sulfate or carboxylate groups; and sulfate anionic surfactants may optionally further comprise one or more carboxylate or sulfonate groups.

Anionic surfactants which may be used include alkyl sulfates, alkyl ether sulfates, alkylamido ether sulfates, alkylaryl polyether sulfates, monoglyceride sulfates, alkyl sulfonates or alpha olefin sulfonates, alkylamide sulfonates, alkylarylsulfonates, paraffin sulfonates, alkyl sulfosuccinates, alkyl ether sulfosuccinates, alkylamide sulfosuccinates, alkyl sulfoacetates, acylsarcosinates, acylglutamates, alkyl sulphosuccinamates, acylisethionates and N-acyl taurates, salts of alkyl monoesters and polyglycosidepolycarboxylic acids, acyl lactylates, N-acyl glycinates, salts of D-galactoside-uronic acids, salts of alkyl ether carboxylic acids, alkyl aryl ether carboxylic acid salts, salts of alkyl amidoether carboxylic acids, sulfoacetates, sulfolaurates, and the corresponding non-salt forms all of these compounds, the alkyl and acyl groups of all these compounds containing from 6 to 40 carbon atoms, especially 14 to 30 carbon atoms, more preferably from 16 to 22 carbon atoms; and aryl means phenyl group. These compounds may be ethoxylated and then preferably comprise from 1 to 50 ethylene oxide units.

Ethylene polyoxyalkylenated (C₆-C₂₄) (amido) ether carboxylic acids and salts thereof may also be cited, in particular those comprising from 2 to 50 alkylene oxide groups, in particular, such as sold by the company KAO under the names AKYPO.

The more preferred alkyl (C₆-C₂₄) (amido) ether carboxylic acids correspond to the following formula:

R₁—(OC₂H₄)ₙ—OCH₂COOA (1)

wherein:
R₁ represents a radical or a mixture of linear or branched alkyl or alkenyl in C₈-C₂₂, a alkyl (C₈-C₉) phenyl radical, a R₂CONH—CH₂—CH₂— group with R₂ denoting an alkyl radical linear or branched alkenyl in C₉-C₂₁; preferably R₁ being an alkyl radical having 8 to 20 carbon atoms, preferably from 8 to 18 carbon atoms and aryl preferably denoting phenyl,
n is an integer or decimal number (average value) which may vary from 2 to 24 and preferably 2 to 10,
A denotes H, ammonium, Na, K, Li, Mg or monoethanolamine or triethanolamine.

It is also possible to use mixtures of compounds of formula (1), in particular mixtures in which the R₁ groups differ.

Polyoxyalkylenated (C₆-C₂₄) (amido) ether carboxylic acids preferably used in the present invention are selected from those of formula (1) wherein:
R₁ denotes a radical or a mixture of (C₁₂-C₁₄)alkyl radicals, cocoyl, oleyl, a nonyl or octylphenyl radical,
A denotes hydrogen or sodium, and
n is from 2 to 20 and preferably 2 to 10.

Polyoxy(C₆-C₂₄)alkylenated ether carboxylic acids and their salts are preferably used, and also polyoxyalkylenated (C₆-C₂₄)alkylamido ether carboxylic acids and salts thereof; in particular those having from 2 to 15 alkylene oxide groups.

Even more preferably, one can use the compounds of formula (1) wherein R is a C12 alkyl radical, A denotes hydrogen or sodium and n is from 2 to 10.

Salts are especially selected from alkali metal salts, especially sodium, ammonium salts, amine salts, amino alcohol such as triethanolamine or monoethanolamine, and magnesium salts.

Preferably, the anionic surfactants are chosen from, alone or as a mixture:
(C₆-C₂₄)alkylsulfates, especially in C₁₂-C₂₀,
(C₆-C₂₄)alkyl ether sulfates, especially in C₁₂-C₂₀, preferably containing from 2 to 20 ethylene oxide units,
(C₆-C₂₄)alkylsulfosuccinates, especially in C12-C20, including laurylsulfosuccinates,
(C₆-C₂₄)alkyl ether sulfosuccinates, especially in C12-C20,
(C₆-C₂₄)acyl sarcosinates, especially in C12-C20, including palmitoylsarcosinates,
(C₆-C₂₄)alkyl ether carboxylates, preferably (C₁₂-C₂₀)alkyl ether carboxylates,
(C₆-C₂₄)acyl isethionates, preferably (C₁₂-C₁₈)acyl isethionates,
polyoxyalkylenated (C₆-C₂₄)alkyl (amido) ether carboxylic acids and salts thereof, in particular those comprising from 2 to 50 alkylene oxide groups, especially ethylene,
(C₆-C₂₄)acylglutamates, especially in C12-C20,
(C₆-C₂₄)acylglycinates, especially in C12-C20,

particularly in the form of alkali or alkaline earth metal, ammonium, amine or aminoalcohol.

More Preferably, the anionic surfactant is chosen from (C₆-C₂₄)alkyl sulfates, (C₆-C₂₄)alkyl ether sulfates such as sodium lauryl ether sulfate (also named sodium laureth sulfate or SLES), isethionates, amino acids, in particular glycinates, such as sodium N-cocoyl glycinate, their alkali salts, and mixtures thereof.

According to a preferred embodiment, the anionic surfactant is a (C₆-C₂₄)alkyl ether sulphate.

According to a more preferred embodiment, the anionic surfactant is sodium lauryl ether sulfate.

Amphoteric and Zwitterionic Surfactants

The composition according to the invention may also comprise one or more amphoteric surfactants.

The amphoteric surfactants that may be used in the invention may be optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

Mention may be made in particular of (C₈-C₂₀)alkylbetaines, sulfobetaines, (C₈-C₂₀)alkylsulfobetaines, (C₈-C₂₀)alkylamido (C₁-C₆)alkylbetaines, such as cocamidopropylbetaine, and (C₈-C₂₀)alkylamido(C₁-C₆)alkylsulfobetaines, and mixtures thereof.

Among the optionally quaternized secondary or tertiary aliphatic amine derivatives that may be used, mention may also be made of the products of respective structures (A1) and (A2) below:

Ra-CON(Z)CH₂—(CH₂)ₘ—N⁺(Rb)(Rc)(CH₂COO⁻) (A1)

in which:

Ra represents a (C₁₀-C₃₀)alkyl or alkenyl group derived from an acid Ra-COOH preferably present in hydrolyzed coconut oil, a heptyl group, a nonyl group or an undecyl group,

Rb represents a β-hydroxyethyl group,

Rc represents a carboxymethyl group,

m is equal to 0, 1 or 2,

Z represents a hydrogen atom or a hydroxyethyl or carboxymethyl group;

R_{a′}—CON(Z)CH₂—(CH₂)_{m′}—N(B)(B′) (A2)

in which:

B represents —CH₂CH₂OX′, with X′ representing —CH₂—COOH, CH₂—COOZ′, —CH₂CH₂—COOH, —CH₂CH₂—COOZ′, or a hydrogen atom,

B′ represents —(CH₂)_{z}—Y′, with z=1 or 2, and Y′ representing —COOH, —COOZ′, —CH₂—CHOH—SO₃H or —CH₂—CHOH—SO₃Z′,

m′ is equal to 0, 1 or 2,

Z represents a hydrogen atom or a hydroxyethyl or carboxymethyl group,

Z′ represents an ion resulting from an alkali or alkaline-earth metal, such as sodium, potassium or magnesium; an ammonium ion; or an ion resulting from an organic amine and in particular from an amino alcohol, such as monoethanolamine, diethanolamine and triethanolamine, monoisopropanolamine, diisopropanolamine or triisopropanolamine, 2-amino-2-methyl-1-propanol, 2-amino-2-methyl-1,3-propanediol and tris(hydroxymethyl)aminomethane,

R_{a′} represents a (C₁₀-C₃₀)alkyl or alkenyl group of an acid R_{a′}COOH preferably present in hydrolyzed linseed oil or coconut oil, an alkyl group, in particular a C17 alkyl group, and its iso form, or an unsaturated C17 group.

The compounds corresponding to formula (A2) are preferred.

Among the compounds corresponding to formula (A2) in which X′ represents an hydrogen atom, mention may be made of compounds classified in the CTFA dictionary, under the names sodium cocoamphoacetate, sodium lauroamphoacetate, sodium caproamphoacetate and sodium capryloamphoacetate.

Compounds corresponding to formula (A2) may be (C₈-C₂₀)alkylamphoacetates and (C₈-C₂₀)alkylamphodiacetates and mixtures thereof.

Other compounds corresponding to formula (A2) are disodium cocoamphodiacetate, disodium lauroamphodiacetate, disodium caproamphodiacetate, disodium capryloamphodiacetate, disodium cocoamphodipropionate, disodium lauroamphodipropionate, disodium caproamphodipropionate, disodium capryloamphodipropionate, lauroamphodipropionic acid and cocoamphodipropionic acid.

Examples that may be mentioned include the cocoamphodiacetate sold by the company Rhodia under the trade name Miranol® C₂M Concentrate, the sodium cocoamphoacetate sold under the trade name Miranol® Ultra C 32 and the product sold by the company Chimex under the trade name Chimexane HA.

Use may also be made of the compounds of formula (A3):

R_{a″}—NH—CH(Y″)—(CH₂)n-C(O)—NH—(CH₂)n′—N(R_{d})(Rₑ) (A3)

in which:
R_{a″} represents a (C₁₀-C₃₀)alkyl or alkenyl group of an acid R_{a″}—C(O)OH preferably present in hydrolysed linseed oil or coconut oil;
Y″ represents the group —C(O)OH, —C(O)OZ″, —CH₂—CH(OH)—SO₃H or the group —CH₂—CH(OH)—SO₃—Z″, with Z″ representing a cationic counterion resulting from an alkali metal or alkaline-earth metal, such as sodium, an ammonium ion or an ion resulting from an organic amine;
R_{d} and Rₑ represent, independently of each other, a (C₁-C₄)alkyl or hydroxyalkyl radical; and
n and n′ denote, independently of each other, an integer ranging from 1 to 3.

Among the compounds corresponding to formula (A3), mention may in particular be made of the compound classified in the CTFA dictionary under the name sodium diethylaminopropyl cocoaspartamide, such as the one sold by the company Chimex under the name Chimexane HB.

Preferably, the amphoteric surfactants are chosen from (C₈-C₂₀)alkylbetaines, (C₈-C₂₀)alkylamido(C₁-C₆)alkylbetaines, (C₈-C₂₀)alkylamphoacetates and (C₈-C₂₀)alkylamphodiacetates, and mixtures thereof.

Nonionic Surfactant

The composition may comprise one or more nonionic alkylpolyglycoside surfactants, especially represented by formula (I):

R₁O—(R₂O)ₜ-(G)ᵥ

wherein:
R₁ represents a linear or branched alkyl or alkenyl radical having 6 to 24 carbon atoms, especially 8 to 18 carbon atoms, or an alkylphenyl radical whose linear or branched alkyl radical comprises from 6 to 24 carbon atoms, especially 8 to 18 carbon atoms,
R₂ represents an alkylene radical having 2 to 4 carbon atoms,
G is a sugar unit containing 5 to 6 carbon atoms,
t is a value ranging from 0 to 10, preferably from 0 to 4,
v is a value ranging from 1 to 15, preferably from 1 to 4.

Preferably, the alkylpolyglycoside surfactants are compounds of formula (I) described above wherein:
R₁ denotes a linear or branched saturated or unsaturated alkyl radical having 8 to 18 carbon atoms,
R₂ represents an alkylene radical having 2 to 4 carbon atoms,
t is a value ranging from 0 to 3, preferably equal to 0,
G denotes glucose, fructose or galactose, preferably glucose,
the degree of polymerization, i.e. the value of v can range from 1 to 15, preferably from 1 to 4; the average degree of polymerization is more particularly between 1 and 2.

Glycosidic linkages between the sugar units are generally 1-6 or 1-4, preferably 1-4.

Preferably, the alkylpolyglycoside surfactant is an alkylpolyglucoside surfactant, even more preferably an C₈-C₁₆ alkylpolyglucosides, and particularly preferably chosen among decylglucosides, caprylyl/capryl glucosides, laurylglucoside, cocoylglucoside, caprylylglucoside, and mixtures thereof.

Among the commercial products, the following product may be cited: products sold by COGNIS under the names PLANTAREN® (600 CS/U, 1200 and 2000) or PLANTACARE® (818, 1200 and 2000); products sold by SEPPIC under the names ORAMIX® CG 110 and ORAMIX® NS 10; products sold by BASF under the name LUTENSOL® GD 70 or products sold by the company CHEM Y under the name AG10 LK®.

Preferably, C₈-C₁₆ alkylpolyglucosides is used, in particular chosen from decylglucoside, caprylyl/capryl glucoside, laurylglucoside, cocoylglucoside, caprylylglucoside, and mixtures thereof.

More preferably, the non ionic surfactant is decylglucoside.

According to a preferred embodiment, the composition may comprise at least one anionic surfactant, preferably chosen from alkyl ether sulfate comprising 6 to 24 carbon atoms, preferably 12 to 20 carbon atoms; at least one non ionic surfactant, preferably chosen from C₈-C₁₆ alkylpolyglucosides; and mixtures thereof.

According to a more preferred embodiment the composition may comprise a surfactant which is chosen among at least one alkyl ether sulfate comprising 6 to 24 carbon atoms, preferably 12 to 20 carbon atoms, more preferably sodium lauryl ether sulfate (SLES); at least one C₈-C₁₆ alkylpolyglucosides, preferably decylglucoside; and mixtures thereof.

In a particular embodiment, the composition comprises sodium lauryl ether sulfate (SLES), and decylglucoside.

Surfactant content in the composition may for example range from 1% to 35% by weight, more preferably from 3% to 30% by weight, still more preferably from 5% to 25% by weight and most preferably from 7% to 20% by weight, relative to the total weight of the composition.

Advantageously, as demonstrated in the experimental part, the amylose deposition on the skin from a film-forming starch suitable for the invention raises with the increase of surfactant content in the cleansing composition according to the invention.

Thus, the surfactant(s) according to the present invention have been found to play a major role on deposition of amylose starch.

Soap

The soap used in the framework of the present invention is an organic soap of fatty acid from 10 to 22 carbon atoms, more preferably from 12 to 18 carbon atoms.

The fatty acid suitable for the present invention may be selected from linear fatty acids, branched fatty acids, and mixtures thereof.

The fatty acid can in particular be selected from caproic acid, capric acid, caprylic acid, oleic acid, linoleic acid, lauric acid, myristic acid, stearic acid, palmitic acid and mixtures thereof.

Preferably, the fatty acid is a linear fatty acid.

According to a preferred embodiment, the fatty acid can be selected from the lauric acid, myristic acid, stearic acid, and mixtures thereof.

The neutralizing agent may be selected from aminoalcohols such as ethanolamine, amino sugars, amino acids, and their alkali salts. Most preferred neutralizing agent is triethanolamine.

The neutralization of soap may be achieved by having a molar ratio between neutralizing agent to fatty acid of at least 1:1.43, preferably of at least 1:1.25.

According to another embodiment, the molar ratio between neutralizing agent to fatty acid ranges from 1:1.43 to 1:1, in particular from 1:1.25 to 1:1.05.

The amount to be taken into consideration for the calculation of the soap amount is the total fatty acid content without the neutralizing agent.

Thus, soap content in the composition may for example range from 5% to 50% by weight, more preferably from 10% to 35% by weight and most preferably from 15% to 25% by weight, relative to the total weight of the composition.

In the present invention, the weight ratio of fatty acid(s) to the surfactant(s) may range from 1.5:1.0 to 5.0:1.0, preferably from 1.6:1.0 to 4.5:1.0, more preferably from 1.7:1.0 to 4.0:1.0.

According to a preferred embodiment, the weight ratio of linear fatty acid(s) to the surfactant(s) may range from 1.5:1.0 to 5.0:1.0, preferably from 1.6:1.0 to 4.5:1.0, more preferably from 1.7:1.0 to 4.0:1.0.

Advantageously, the presence of both surfactants and soap in a cleansing composition according to the invention imparts foam during product application and removal of impurities like particulates, dirt and sebum from skin.

Furthermore, presence of both soap and surfactant(s) gives a balance between big bubbles open foam and creamier foam in addition to quick rinse and feeling clean after application of the composition.

Additives

The composition according to the invention may contain various additives, chosen from those conventionally used in skincare products, insofar as these additives and the amounts thereof do not harm the qualities desired for the composition according to the invention.

The cleansing composition in accordance with the present invention may thus comprise the following additives: sequestrants (e.g. EDTA and salts thereof); antioxidants (e.g. butylated hydroxytoluene also named BHT); biological extracts; antibacterial agents, fragrances (e.g. perfumes, essential oils); dyestuffs; encapsulated or non-encapsulated pigments or soluble dyes; thickeners (e.g. glycol distearate); preservatives (e.g. phenoxyethanol, methylisothiazolinone).

The amounts of these various adjuvants are those conventionally used in the field under consideration, for example from 0.01% to 20% of active material of the total weight of the composition. These adjuvants and the amounts thereof should be such that they do not modify the property desired for the composition of the invention.

Composition

The composition according to the invention comprises an aqueous medium or aqueous phase, i.e. a medium comprising an amount of water ranging from 35% to 80% by weight, preferably from 40% to 70% by weight and more preferably from 43% to 60% by weight relative to the total weight of the composition.

The aqueous phase of the compositions according to the invention may contain, besides water, one or more water soluble solvents at room temperature (25° C.), such as for example polyols with 2 to 20 carbon atoms, and mixtures thereof.

For the purpose of the present invention, the term “polyol” should be understood to mean any organic molecule comprising at least two free hydroxyl groups.

A polyol suitable for the invention may be a compound such as a saturated or unsaturated, linear, branched or cyclic alkyl bearing, on the alkyl chain, at least two —OH functions, in particular at least three —OH functions, and more particularly at least four —OH functions.

The polyols advantageously suitable for the formulation of the cleansing composition according to the present invention are those having, in particular, from 2 to 20 preferably 2 to 16 carbon atoms, preferably 2 to 10, preferably 3 to 8 carbon atoms.

Among polyols, the following may be cited: glycerine, 1,3-propanediol, isoprene glycol, pentylene glycol, hexylene glycol, glycols such as ethylene glycol, propylene glycol, butylene glycol, diethylene glycol and dipropylene glycol, polyglycerols with 2 to 6 repeating units, for instance diglycerol, erythritol, arabitol, adonitol, sorbitol, dulcitol, glucose, fructose, xylose, trehalose, sucrose, maltose, saccharose and lactose, and mixtures thereof.

According to a preferred embodiment, the polyol is selected from glycerine, sorbitol, and mixtures thereof. More preferably, the polyol is selected from glycerine and sorbitol.

When they are present, the amount of polyols in the composition of the invention may range, for example, from 0.1% to 20% by weight, preferably from 0.5% to 15% by weight and better still from 5% to 15% by weight relative to the total weight of the composition.

Typically, the polyols act as moisturizing agents which can deliver better skin smoothness and skin moisture balance.

Throughout the description, including the claims, the expression “comprising a” should be understood as being synonymous with “comprising at least one”, unless otherwise specified.

The expressions “between . . . and . . . ” and “ranging from . . . to . . . ” should be understood as meaning limits included, unless otherwise specified.

The examples that follow illustrate the present invention without limiting the scope thereof.

The cleansing compositions described in the following examples are prepared using the following general process:
1. Water and polyol(s) are mixed and stirred for 30 minutes.
2. Chelating agents and pigments (if any, for colored product) are added and heated until 70 or 80° C., for example 75° C.
3. Then, fatty acids are added followed by surfactants and allowed for complete melting and mixing.
4. Soap neutralizer is added.
5. Starch is then added slowly to ensure complete dispersion and is stirred for 30 to 45 minutes.
6. Cationic polymer is added.
7. Then, the whole mixture is cooled to attain the room temperature (25° C.).
8. Optional ingredients (such as preservative, extract, antioxidant, fragrance etc.) in connection to the texture of the product are then added at room temperature.
9. After addition of each component, mixing is continued for 20 to 40 minutes until homogeneous mixture is obtained.

Ingredient amounts are indicated in the following examples in active material weight percentages “% wgt”.

### EXAMPLES

Preliminarily, it has to be noted that the term «comp» in the following tables means that the corresponding composition is outside the scope of the present invention and the term «inv» means that the corresponding composition illustrates the invention as claimed.

### Product Evaluation

Performance of the products was measured in terms of amylose deposition on skin and skin whitening, as well as in terms of sebum level on skin and long lasting oil control through the formulations.

### 1. Amylose Deposition:

Amylose deposition was measured by reflectance (ATR) FTIR spectroscopy.

Symmetric stretching of C—O—C bond of amylose at 1026 cm⁻¹ was considered as peak of interest.

Area under (a.u.) the curve (corresponding to the peak at 1026 cm⁻¹) was considered as signature of amylose and was measured for hand washed with water or product using a fixed protocol.

Then, normalized area (corrected with peak of hand washed with water) was considered as signature of amylose deposition on skin. Higher value area under the curve represents higher amylose deposition.

### Protocol for Cleanser Product Application on Hand:

One of the forearms of human volunteers has been wet. Then, 1 g product was applied (Temperature of 21° C. and RH of 50±5%) and rubbed on to the skin in circular motion (10 times repeat) covering the entire forearm region. Then, the skin was rinsed-off with water (during 30 seconds) followed by drying. For the drying, the skin was dabbed with a dry tissue which was gently placed on the arm to absorb water.

Similar protocol followed for the hand washing with water.

Each experiment was conducted in triplicates on same volunteer with each of the cleanser product and average of that is reported to the corresponding example.

Amylose deposition through product formulae 4 and 5 were performed with 10 different volunteers to check the variation of amylose deposition with skin type. For the other formulae, there were 3 volunteers for each formula.

Standard deviation for all experiments ranges from 0.0005 to 0.02. This indicates repeatability and accuracy of the method.

### 2. Face Sebum Evaluation:

First, face was washed with only water using standard gesture for skin normalization, followed by 30 minutes waiting under controlled conditions (Temperature of 21° C. and RH of 50±5%).

Cleanser product of 1 g was applied on full face using a fixed protocol followed by rinsing and drying.

Finally sebum level at different time frame (after wash and before wash) was measured using sebumeter.

The face of 16 human volunteers was treated with five cleanser products. Sebum level was measured using sebumeter at different time frame before and after face wash.

Lower percentage of sebum evolution [(Sebum at T=2 hours−Sebum at T=0 minutes)/Sebum at T=0 minutes] indicates better oil control.

### Protocol for Cleanser Product Application:

Sebum level measurements were performed with 16 different volunteers.

One measurement was done at each time point for a volunteer.

First, hand was washed properly under running water.

The face of human volunteer was dampened with wet hand with downward motion and same was repeated twice.

Then, product was applied (0.5 g for half face) uniformly using the same hand with a specific gesture, moving from forehead to chin and again back to forehead. This was repeated 10 times.

Then, the hands were washed under running water till the entire product was removed.

More particularly, the hands are rinsed under tap water for 2 seconds, and the wet hands (water in palms is not reserved) are moved on face from chin to forehead and back to chin (2 sides of face-7×cycles). Each time the hand is rinsed in between washes by keeping hand under running water for 2 seconds.

The face was dabbed with dry tissue paper which was gently placed (without rubbing) to absorb water from face.

### Example 1: Effect of the Amylose Content in Starch

Formula 1 is a comparative composition as it does not comprise a film-forming starch suitable for the present invention whereas formulae 2 and 3 are according to the present invention. More particularly, in the comparative formula 1, a starch which does not contain amylose (that is to say, amylose content: 0% by weight with respect to the total weight of starch) was tested in view of two film-forming starches suitable for the invention which respectively contain 40% (formula 2) and 70% (formula 3) by weight of amylose with respect to the total weight of starch.

Table 1 states the measurements of the amylose deposition for these three formulae.

| | Formula 1 | Formula 2 | Formula 3 |
|---|---|---|---|
| INCI Name | (comp) | (inv) | (inv) |
| Water | q.s. 100 | q.s. 100 | q.s. 100 |
| Sorbitol | 7.00 | 7.00 | 7.00 |
| Disodium EDTA | 0.26 | 0.26 | 0.26 |
| Myristic Acid | 5.44 | 5.44 | 5.44 |
| Lauric Acid | 10.00 | 10.00 | 10.00 |
| Stearic Acid | 4.00 | 4.00 | 4.00 |
| Decyl Glucoside | 1.30 | 1.30 | 1.30 |
| Sodium Laureth Sulfate sold under | 7.00 | 7.00 | 7.00 |
| the name of GALAXY ® LES170 | | | |
| by Galaxy surfactants | | | |
| Glycol Distearate | 3.00 | 3.00 | 3.00 |
| Triethanolamine | 2.00 | 2.00 | 2.00 |
| Potassium Hydroxide | 3.70 | 3.70 | 3.70 |
| Hydroxypropyl Corn Starch | 10.00 | — | — |
| Phosphate sold under the name of | | | |
| Structure ® ZEA by Akzo Nobel | | | |
| (amylose content: 0%) | | | |
| Hydroxypropyl Pea | — | 10.00 | — |
| Starch sold under the name | | | |
| of Lycoat ® RS 720 by | | | |
| Roquette (amylose content: 40%) | | | |
| Hydroxypropyl Corn Starch | — | — | 10.00 |
| Modified sold under the name of | | | |
| Amaze ® by Akzo Nobel (amylose | | | |
| content: 70%) | | | |
| Polyquaternium-67 sold under the | 0.40 | 0.40 | 0.40 |
| name of | | | |
| SOFTCAT ® POLYMER SL-100 | | | |
| by Dow Chemical | | | |
| BHT | 0.05 | 0.05 | 0.05 |
| Phenoxyethanol | 0.20 | 0.20 | 0.20 |
| Fragrance | 0.40 | 0.40 | 0.40 |

TABLE 1

| | Amylose | | Amylose |
|---|---|---|---|
| | deposition | | Content |
| Formula | (a.u.) | Starch Chemical Name | (%) |
| Formula 1 | 0.034 | Hydroxypropyl Corn Starch | 25 |
| (comp) | | Phosphate | |
| Formula 2 | 0.07 | Hydroxypropyl Pea Starch | 40 |
| (inv) | | | |
| Formula 3 | 0.08 | Hydroxypropyl Corn Starch | 70 |
| (inv) | | Modified | |

These results indicate that amylose deposition of starch on skin raises with the increase of amylose content in the starch in the joint presence of Polyquaternium-67 (cationic polymer), soaps and surfactants.

Cleansing composition of formulae 2 and 3 in accordance with the invention provide good whitening properties thanks to the amylose deposition on the skin.

### Example 2: Effect of the Cationic Polymer on Amylose Deposition

Formula 5 is a comparative composition as it does not contain cationic polymer suitable for the present invention whereas formulae 4 and 6 to 9 are part of the present invention.

Formula 2 as illustrated in example 1 is also tested (see table 2 below).

Table 2 states the measurements of the amylose deposition for these seven formulae.

| | Formula 4 | Formula 5 | Formula 6 | Formula 7 | Formula 8 | Formula 9 |
|---|---|---|---|---|---|---|
| INCI Name | (inv) | (comp) | (inv) | (inv) | (inv) | (inv) |
| Water | q.s. 100 | q.s. 100 | q.s. 100 | q.s. 100 | q.s. 100 | q.s. 100 |
| Sorbitol | 7.00 | 7.00 | 7.00 | 7.00 | 7.00 | 7.00 |
| Disodium EDTA | 0.26 | 0.26 | 0.3 | 0.3 | 0.3 | 0.30 |
| Titanium dioxide | — | — | 0.5 | 0.5 | 0.5 | 0.50 |
| Myristic acid | 5.44 | 5.44 | 5.5 | 5.5 | 5.5 | 5.50 |
| Lauric acid | 10.00 | 10.00 | 10.00 | 10.00 | 10.00 | 10.00 |
| Stearic acid | 4.00 | 4.00 | 4.00 | 4.00 | 4.00 | 4.00 |
| Glycol distearate | 3.00 | 3.00 | 3.00 | 3.00 | 3.00 | 3.00 |
| Decyl glucoside | 1.30 | 1.30 | 1.30 | 1.30 | 1.30 | 1.30 |
| Sodium laureth | 7.00 | 7.00 | 7.00 | 7.00 | 7.00 | 7.00 |
| sulfate sold under | | | | | | |
| the name of. | | | | | | |
| GALAXY ® LES170 | | | | | | |
| by Galaxy surfactants | | | | | | |
| Triethanolamine | 2.00 | 2.00 | 2.00 | 2.00 | 2.00 | 2.00 |
| Potassium | 3.70 | 3.70 | 3.70 | 3.70 | 3.70 | 3.70 |
| hydroxide | | | | | | |
| Hydroxypropyl pea | 10.00 | 10.00 | 10.00 | 10.00 | 10.00 | 10.00 |
| starch sold under | | | | | | |
| the name of | | | | | | |
| Lycoat ® RS 720 by | | | | | | |
| Roquette (amylose | | | | | | |
| content: 40%) | | | | | | |
| Polyquaternium-4 | — | — | — | 0.40 | — | — |
| sold under the | | | | | | |
| name of | | | | | | |
| CELQUAT ® LOR | | | | | | |
| by Akzo Nobel | | | | | | |
| Hydroxypropyl | — | — | — | — | — | 0.40 |
| guar hydroxypropyl | | | | | | |
| trimonium chloride | | | | | | |
| sold under the | | | | | | |
| name of | | | | | | |
| JAGUAR ® C 162 | | | | | | |
| by Rhodia. | | | | | | |
| Polyquaternium-7 | 0.46 | — | — | — | — | — |
| sold under the | | | | | | |
| name of | | | | | | |
| MIRAPOL ® 550 S | | | | | | |
| BO by Rhodia | | | | | | |
| Polyquaternium-39 | — | — | 0.40 | — | — | — |
| sold under the | | | | | | |
| name of | | | | | | |
| MERQUAT ® 3330 | | | | | | |
| PR POLYMER by | | | | | | |
| Lubrizol. | | | | | | |
| Polyquaternium-53 | — | — | — | — | 0.40 | — |
| sold under the | | | | | | |
| name of | | | | | | |
| MERQUAT ® 2003 | | | | | | |
| PR POLYMER by | | | | | | |
| Lubrizol | | | | | | |
| BHT | 0.05 | 0.05 | 0.05 | 0.05 | 0.05 | 0.05 |
| Phenoxyethanol | 0.20 | 0.20 | 0.20 | 0.20 | 0.20 | 0.20 |
| Fragrance | 0.40 | 0.4 | 0.4 | 0.40 | 0.40 | 0.40 |

TABLE 2

| | | Formula | Formula | Formula | Formula | Formula | Formula | Formula |
|---|---|---|---|---|---|---|---|---|
| | Blank | 4 | 5 | 2 | 6 | 7 | 8 | 9 |
| Formula | (water) | (inv) | (comp) | (inv) | (inv) | (inv) | (inv) | (inv) |
| Amylose | 0.018 | 0.094 | 0.046 | 0.072 | 0.052 | 0.070 | 0.076 | 0.06 |
| Deposition | | | | | | | | |
| (a.u.) | | | | | | | | |

These results show that amylose deposition from hydroxypropyl pea starch (amylose content: 40%) in the presence of soaps and surfactant is higher in the presence of cationic polymers (Formulae 2, 4, 6, 7, 8 and 9 according to the invention) compared to without any cationic polymer (comparative Formula 5).

Cleansing composition of formulae 2, 4 and 6 to 9 in accordance with the invention provide good whitening properties.

### Example 3: Effect of Soap and Surfactant on Amylose Deposition

Formula 10 and formula 13 are comparative compositions as they do not respectively contain surfactant and soap suitable for the present invention whereas formulae 11 and 12 belong to the present invention.

Table 3 states the measurements of the amylose deposition for these four formulae as well as for a composition exclusively made of water (blank).

| | Formula 10 | Formula 11 | Formula 12 |
|---|---|---|---|
| INCI NAME | (comp) | (inv) | (inv) |
| Water | q.s. 100 | q.s. 100 | q.s. 100 |
| Glycerin | 9.00 | 9.00 | 9.00 |
| Titanium dioxide | 0.50 | 0.50 | 0.50 |
| Disodium EDTA | 0.30 | 0.30 | 0.30 |
| Myristic acid | 10.00 | 7.60 | 6.40 |
| Lauric acid | 10.00 | 7.60 | 6.40 |
| Stearic acid | 9.00 | 6.80 | 5.70 |
| Sodium laureth sulfate | — | 7.00 | 10.50 |
| sold under the name of | | | |
| GALAXY ® LES170 | | | |
| by Galaxy | | | |
| surfactants | | | |
| Glycol distearate | 3.00 | 3.00 | 3.00 |
| Potassium hydroxide | 6.65 | 5.05 | 4.25 |
| Hydroxypropyl Corn | 4.00 | 4.00 | 4.00 |
| starch modified sold | | | |
| under the name of | | | |
| AMAZE ® by Akzo | | | |
| Nobel (amylose | | | |
| content: 70%) | | | |
| Polyquaternium-7 sold | 0.46 | 0.46 | 0.46 |
| under the name of | | | |
| MIRAPOL ® 550 SBO | | | |
| by Rhodia | | | |
| BHT | 0.05 | 0.05 | 0.05 |
| Phenoxyethanol | 0.50 | 0.50 | 0.50 |
| Methylisothiazolinone | 0.01 | 0.01 | 0.01 |
| Fragrance | 0.40 | 0.40 | 0.40 |

TABLE 3

| | | Formula | Formula | Formula |
|---|---|---|---|---|
| | Blank | 10 | 11 | 12 |
| Formula | (water) | (comp) | (inv) | (inv) |
| Amylose Deposition (a.u) | 0.018 | 0.046 | 0.082 | 0.093 |

From these results, it comes out that in the presence of cationic polymer (Polyquaternium-7), amylose deposition from hydroxypropyl corn starch modified (70% amylose) on skin raises with the increase of surfactant content in the formulations.

Cleansing composition of formulae 11 and 12 in accordance with the invention provide good whitening properties.

### Example 4: Effect of Amylose on Sebum Evolution

Formula 13 is a comparative composition as it does not comprise film-forming starch and cationic polymer suitable for the present invention whereas formulae 14 and 15 are according to the present invention.

Table 4 states the percentage of sebum evolution by comparing the values which are measured, on one hand, before wash (sebum at T=0 minute) and, on the other hand, after wash (sebum at 10 minutes and 2 hours).

These measurements are carried out for formulae 2 as illustrated in example 1 above, 13, 14, 15, and for a formula consisting of water exclusively (blank).

| | Formula | Formula | Formula |
|---|---|---|---|
| | 13 | 14 | 15 |
| INCI NAME | (comp) | (inv) | (inv) |
| Water | q.s. 100 | q.s. 100 | q.s. 100 |
| Sorbitol | 7.00 | 7.00 | 7.00 |
| Disodium EDTA | 0.26 | 0.30 | 0.30 |
| Myristic acid | 5.44 | 5.50 | 5.50 |
| Laurie acid | 10.00 | 10 | 10 |
| Stearic acid | 4.00 | 4.00 | 4.00 |
| Decyl glucoside | 2.50 | 2.50 | 2.50 |
| Sodium laureth sulfate sold under the | 7.00 | 7.00 | 7.00 |
| name of GALAXY ® LES170 by | | | |
| Galaxy surfactants | | | |
| Glycol distearate | 3.00 | 3.00 | 3.00 |
| Triethanolamine | 2.00 | 2.00 | 2.00 |
| Potassium hydroxide | 3.70 | 3.70 | 3.70 |
| Hydroxypropyl Corn starch modified | — | 10.00 | 6.00 |
| sold under the name of AMAZE ® by | | | |
| Akzo Nobel (amylose content: 70%) | | | |
| Polyquaternium-67 sold under the | — | 0.40 | 0.40 |
| name of | | | |
| SOFTCAT ® POLYMER SL-100 by | | | |
| Dow Chemical | | | |
| BHT | 0.05 | 0.05 | 0.05 |
| Phenoxyethanol | 0.20 | 0.20 | 0.20 |
| Fragrance | 0.40 | 0.40 | 0.40 |

Protocol

The in vivo tests are as follows.

The face of 16 human volunteers was treated with five cleanser products, that is to say with formulae 2, 13, 14, 15, and water.

Sebum level was measured using sebumeter at different time frame before and after face wash.

Lower percentage of sebum evolution [(Sebum at T=2 hours−Sebum at T=0 minutes)/Sebum at T=0 minutes] indicates better oil control.

### Results and Conclusion

TABLE 4

| | | | | Percentage of |
|---|---|---|---|---|
| | Before wash | After wash | | Sebum |
| Formula | Sebum at | Sebum at | Sebum at | Evolution |
| Number | T = 0 minute | T = 10 minutes | T = 2 hours | after 2 hours |
| Water (blank) | 79.9 | 69.0 | 130.7 | 63.6 |
| Formula 13 | 79.4 | 63.6 | 117.6 | 48.1 |
| (comp) | | | | |
| Formula 2 | 78.9 | 57.6 | 112.1 | 42.0 |
| (inv) | | | | |
| Formula 14 | 80.3 | 61.3 | 112.6 | 40.2 |
| (inv) | | | | |
| Formula 15 | 82.0 | 67.8 | 115.4 | 40.7 |
| (inv) | | | | |

From the results shown in table 4, it comes out that sebum evolution is higher in the absence of film-forming starch and cationic polymer (see formula 13 and water treatment). Progressively, in the presence of cationic polymer, sebum evolution is slower for film-forming starch containing compositions (see Formulae 2, 14, and 15).

Based on the whole above results (Tables 1, 2, 3, and 4) and examples, it comes out that cationic polymer raises the deposition of amylose from the film-forming starch on skin in the presence of both soap and surfactant.

Moreover, in the joint presence of cationic polymer, soap and surfactant, amylose deposition on skin raises with the increase of amylose content in the film-forming starch.

Further, in the presence of cationic polymer, amylose deposition on skin from a film-forming starch raises with the increase of surfactant content in the formulations.

Thus, skin cleansing with cleansing compositions according to the present invention results into amylose deposition on skin, which turns into better sebum control of skin and skin whitening after face wash and/or body wash.

In conclusion, cleansing composition in accordance with the invention provides both good whitening and oil control properties after rinse off.

### Example 5: Intrinsic Power of Starch Deposition Between Film-Forming Starches According to the Invention and a Comparative Starch, as Well as Viscosity Measurements

Films using aqueous solution comprising 10% by weight of starch (10% solution) of three different starches were formed using BYK Automatic Film Applicator on Polyethylene sheet. The film thickness was about 25 microns (1 mil).

Then, these starches were full dried and left for two days before performing the visual observations.

In this example, two starches are suitable for the invention (Hydroxypropyl Pea Starch (amylose content: 40%) and Hydroxypropyl Corn Starch Modified (amylose content: 70%) and one starch is outside the invention (Hydroxypropyl Corn Starch phosphate (amylose content: 0%).

Furthermore, the viscosity of each solution was measured at 25° C. and atmospheric pressure using a Rheomat RM180® apparatus. The viscosity was measured using mobile 2 (for the film-forming starches suitable for the invention) or mobile 4 (for the comparative starch) attachment to Rheomat RM180® at a rotation speed of 200 (fixed) after rotation of the measuring instrument for 10 minutes. The corresponding value in UD (unit deflection) is converted to mPa·s (cps).

The visual observations and the viscosity measurements are gathered in the following table 5.

TABLE 5

| | VISCOSITY | VISUAL |
|---|---|---|
| | (mPa · s) at 10 | OBSER- |
| STARCHES | min/mobile | VATIONS |
| Hydroxypropyl Pea Starch (amylose | <37.5/mobile | very transparent |
| content: 40%) sold under the name of | 2 | film with high |
| Lycoat ® RS 720 by Roquette Company | | uniformity and |
| (According to the invention) | | homogeneity |
| Hydroxypropyl Corn Starch Modified | 224/mobile 2 | very good film |
| (amylose content : 70%) sold under the | | with high |
| name of AMAZE ® Starch by Akzo | | uniformity and |
| Nobel Company | | homogeneity |
| (According to the invention) | | |
| Hydroxypropyl Corn Starch phosphate | 6 665/mobile | Film not regular |
| (amylose content : 0%) sold under the | 4 | and not |
| name of Structure ® Zea by Akzo Nobel | | uniform |
| Company | | |
| (Comparatinve) | | |

It comes out from this example that film-forming starches suitable for the present invention are able to form very transparent and good films on polyethylene sheets with high uniformity and homogeneity whereas starches outside of the invention present a film which is not regular and not uniform.

1: A cleansing composition comprising, in a physiologically acceptable aqueous medium:
(a) at least a film-forming starch,
(b) at least one cationic polymer,
(c) at least one surfactant, and
(d) at least a soap.

2: The composition according to claim 1, wherein the film-forming starch is a starch which contains an amylose content in an amount higher than or equal to 40% by weight, with respect to the total weight of starch.

3: The composition according to claim 1, wherein the film-forming starch is selected from the group consisting of hydroxypropyl pea starch with an amylose content of 40% by weight with respect to the total weight of starch, hydroxypropyl corn starch modified with an amylose content of 70% by weight with respect to the total weight of starch, and mixtures thereof.

4: The composition according to claim 1, wherein the film-forming starch is present in the composition in an amount from 3% to 12% by weight, with respect to the total weight of the composition.

5: The composition according to claim 1, wherein the film-forming starch has a viscosity, measured at 25° C. and atmospheric pressure, which ranges from 20 to 400 mPa·s, of an aqueous solution comprising 10% by weight of said starch.

6: The composition according to claim 1, wherein the cationic polymers are chosen from Polyquaternium-4, Polyquaternium-7, Polyquaternium-53, Polyquaternium-67, hydroxypropyl guar hydroxypropyl trimonium chloride, and mixtures thereof.

7: The composition according to claim 1, wherein the cationic polymer is present in the composition in an amount ranging from 0.3% to 0.6% by weight relative to the total weight of the composition.

8: The composition according to claim 1, wherein the weight ratio of film-forming starch to the cationic polymer ranges from 5:1 to 40:1.

9: The composition according to claim 1, wherein the surfactant is chosen among anionic surfactants, amphoteric surfactants, nonionic surfactants, and mixtures thereof.

10: The composition according to claim 1, wherein the surfactant is chosen among at least one alkyl ether sulfate comprising 6 to 24 carbon atoms; at least one C₈-C₁₆ alkylpolyglucosides; and mixtures thereof.

11: The composition according to claim 1, wherein the surfactant is present in the composition in an amount ranging from 1% to 35% by weight, relative to the total weight of the composition.

12: The composition according to claim 1, wherein the soap is an organic soap of fatty acid from 10 to 22 carbon atoms.

13: The composition according to claim 12, wherein the fatty acid is selected from caproic acid, capric acid, caprylic acid, oleic acid, linoleic acid, lauric acid, myristic acid, stearic acid, palmitic acid and mixtures thereof.

14: The composition according to claim 1, wherein the soap is present in the composition in an amount ranging from 5% to 50% by weight, relative to the total weight of the composition.

15: The composition according to claim 1, wherein the weight ratio of linear fatty acid to the surfactant ranges from 1.5:1.0 to 5.0:1.

16: The composition according to claim 1, wherein water is present in an amount ranging from 35% to 80% by weight, relative to the total weight of the composition.

17: A process for cleansing the skin, consisting of:
applying a composition to the skin; and
applying water, in working the composition into a foam and then in rinsing off the composition,
wherein the composition comprises:
(a) at least a film-forming starch,
(b) at least one cationic polymer,
(c) at least one surfactant, and
(d) at least a soap.
